(19) United States
(12) Patent Application Publication
(10) Pub. No.: US 2025/0125033 A1
(43) Pub. Date: Apr. 17, 2025

(54) Method for Training Model, Method for Generating Treatment Plan, and Medium

(71) Applicants: OUR UNITED CORPORATION, Shaanxi (CN); SHENZHEN OUR NEW MEDICAL TECHNOLOGIES DEVELOPMENT CO., LTD., Shenzhen (CN); OUR SMD Medical (Wuhan) Co., Ltd., Wuhan (CN)

(72) Inventors: Peng Guo, Shaanxi (CN); Jinsheng Li, Shaanxi (CN)

(21) Appl. No.: 18/498,772

(22) Filed: Oct. 31, 2023

(30) Foreign Application Priority Data
Oct. 16, 2023 (CN) 202311339042.3

(51) Int. Cl. G16H 20/40; G06N 3/092

(52) U.S. Cl. CPC G16H 20/40; G06N 3/092

(57) ABSTRACT

Methods for training a deep reinforcement learning model for generating a treatment plan and an electronic device are provided in the present disclosure. The method may include performing a training process. The training process includes obtaining initial dose distribution state data of an objective target volume; determining target data based on the initial dose distribution state data of the objective target volume, current policy data of a plurality of actor network layers, and current policy data of a critic network layer; and completing the current training by updating the current policy data of the plurality of actor network layers and the current policy data of the critic network layer based on the target data; and obtaining a trained deep reinforcement learning model by iterating the training process until a count of iterating the training process reaches a preset value.

### CROSS-REFERENCE TO RELATED APPLICATION

This application claims priority to Chinese Patent Application No. 202311339042.3, filed Oct. 16, 2023, the disclosure of which is hereby incorporated by reference in its entirety.

### BACKGROUND OF THE INVENTION

### Field of the Invention

The present disclosure relates to the field of medical technology, and in particular, to the field of radiotherapy, for example, to a method for training a model, a method for generating a treatment plan, and a medium.

### Description of Related Art

In the field of medical technology, radiotherapy is one of the important means to treat tumors. Before using radiotherapy for treating a to-be-treated subject, it is usually necessary to design a treatment plan in advance.

At present, the design for a more reasonable treatment plan usually relies on a physician to repeatedly adjust the treatment plan manually based on their own experience and professional skills. Therefore, the existing methods for generating a treatment plan require a high level of clinical experience from the physicians, and the treatment plan requires continuous trial and error, which is time-consuming, laborious and inefficient.

### SUMMARY OF THE INVENTION

The present disclosure provides a method for training a model, a method for generating a treatment plan, a device and a medium.

One aspect of the present disclosure provides a method for training a deep reinforcement learning model for generating a treatment plan. The deep reinforcement learning model is configured to include a plurality of actor network layers and a critic network layer, and different actor network layers of the plurality of actor network layers are configured to output different types of target parameters included in the treatment plan, the method includes: performing a training process, the training process including the following operations: acquiring initial dose distribution state data of an objective target volume; determining, based on the initial dose distribution state data of the objective target volume, current policy data of the plurality of actor network layers, and current policy data of the critic network layer, target data; and updating, based on the target data, the current policy data of the plurality of actor network layers and the current policy data of the critic network layer, so as to complete a current training for the deep reinforcement learning model; and iterating the training process until a count of training the deep reinforcement learning model reaches a preset count, so as to obtain the deep reinforcement learning model that has been trained;

where the target data includes: a final dose distribution of the objective target volume, multiple dose distribution state data of the objective target volume, a plurality of action sets output by the plurality of actor network layers corresponding to the multiple dose distribution state data respectively, a plurality of predicted values output by the critic network layer corresponding to the plurality of action sets respectively, a plurality of actual rewards corresponding to the plurality of action sets respectively, a predicted value of the objective target volume, and an actual reward of the objective target volume; where each action set of the plurality of action sets includes a target parameter combination composed of a plurality of different types of target parameters.

Another aspect of the present disclosure provides a method for generating a treatment plan. The method includes: acquiring image data of a to-be-treated target volume and contour data of the to-be-treated target volume; determining, based on the image data and the contour data, dose distribution state data of the to-be-treated target volume; inputting the dose distribution state data of the to-be-treated target volume into a deep reinforcement learning model, so as to obtain a target parameter combination composed of a plurality of different types of target parameters of the to-be-treated target volume; where the deep reinforcement learning model is trained by the method for training the deep reinforcement learning model for generating a treatment plan according to the above aspect.

Yet another aspect of the present disclosure provides an electronic device. The electronic device includes at least one processor, and a storage connected with the at least one processor. The storage stores an instruction that can be executed by the at least one processor, when the instruction is executed by the at least one processor, causing the at least one processor to implement the method for training the deep reinforcement learning model according to the above aspect.

Yet another aspect of the present disclosure provides a non-transitory computer readable storage medium having stored a computer program thereon, where the program, upon being executed by a processor, implements the method for training the deep reinforcement learning model according to the above aspect.

In the embodiments of the present disclosure, a method for training a deep reinforcement learning model for generating a treatment plan, a method for generating a treatment plan, a device, and a medium are provided. The deep reinforcement learning model is configured to include a plurality of actor network layers and a critic network layer, and different actor network layers of the plurality of actor network layers are configured to output different types of target parameters included in the treatment plan. Thus, after acquiring the initial dose distribution state data of the objective target volume, the target data is determined based on the initial dose distribution state data of the objective target volume, current policy data of the plurality of actor network layers, and current policy data of the critic network layer. Then, the current policy data of the plurality of actor network layers and the current policy data of the critic network layer may be updated based on the target data, so as to complete the current training for the deep reinforcement learning model. The training process is iterated until a count of training the deep reinforcement learning model reaches a preset count, so as to obtain the deep reinforcement learning model that has been trained.

The target data includes: a final dose distribution of the objective target volume, multiple dose distribution state data of the objective target volume, a plurality of action sets output by the plurality of actor network layers corresponding to the multiple dose distribution state data respectively, a plurality of predicted values output by the critic network layer corresponding to the plurality of action sets respectively, a plurality of actual rewards corresponding to the plurality of action sets respectively, a predicted value of the objective target volume, and an actual reward of the objective target volume; where each action set of the plurality of action sets includes a target parameter combination composed of a plurality of different types of target parameters.

As set forth above, an overall task of determining action sets for different types of target parameters may be divided into a plurality of subtasks for determining actions for different types of target parameters in the present disclosure. Each actor network layer of the plurality of actor network layers in the deep reinforcement learning model completes a subtask, that is, the complex overall task is divided into the plurality of subtasks for calculation. Therefore, the plurality of actor network layers may improve the efficiency of determining target parameters. Due to the fact that the deep reinforcement learning model conforms to the characteristics of the design for the treatment plan with Gamma knife, and a processor in a computer system is able to repeat the trial and error process, target parameters with better performance may be automatically generated through the deep reinforcement learning model, resulting in generating a better treatment plan based on the target parameters with better performance. Thus, the dependence on clinical experience may be reduced, the effect of treatment plan may be improved without the need for manual setting of target parameters, and further, the efficiency for generating a treatment plan by a physician is improved.

It should be understood that the contents described in the section is not intended to identify keys or important features of the embodiments of the present disclosure, nor is it used to limit the scope of the present disclosure. The other features of the present disclosure will be easily understood through the following description.

### BRIEF DESCRIPTION OF THE DRAWINGS

The drawings are intended for a better understanding of the present disclosure, and do not constitute the limitation of the present disclosure.

FIG. 1 is a schematic diagram of an exemplary application scenario of a method for training a deep reinforcement learning model for generating a treatment plan, and a method for generating a treatment plan according to some embodiments.

FIG. 2 is a flow chart of an exemplary method for training a deep reinforcement learning model according to some embodiments.

FIG. 3 is another flow chart of an exemplary method for training a deep reinforcement learning model according to some embodiments.

FIG. 4 is a flow chart of an exemplary method for generating a treatment plan according to some embodiments.

FIG. 5 is a block diagram of an electronic device for implementing embodiments of the present disclosure according to some embodiments.

### DESCRIPTION OF THE INVENTION

Exemplary embodiments of the present disclosure are described below with reference to the drawings, in which various details of the embodiments of the present disclosure are included to facilitate understanding, and these embodiments should be considered as exemplary only. Therefore, those skilled in the art should realize that various changes and modifications can be made to the embodiments described herein without departing from the scope of the present disclosure. Similarly, for clarity and conciseness, description of well-known functions and structures have been omitted from the following description.

In the technical solution in the present disclosure, the collection, storage, usage, processing, transmission, provision, and disclosure of user personal information comply with relevant laws and regulations, and do not violate public order and good customs.

Before a detailed description on the present disclosure is provided, the application scenarios involved in the embodiments of the present disclosure are described first.

A method for training a deep reinforcement learning model for generating a treatment plan, a method for generating a treatment plan, a device, and a medium provided in the embodiments of the present disclosure may be applied in the field of medical technology, and in particular, in the scenarios of the clinical radiotherapy, such as a scenario for designing a treatment plan with Gamma knife.

Before performing radiotherapy on a to-be-treated subject, it is usually necessary to design a treatment plan in advance.

At present, the design for a more reasonable treatment plan usually relies on a physician to repeatedly adjust the treatment plan manually based on their own experience and professional skills. Therefore, the existing methods for generating a treatment plan require a high level of clinical experience from the physicians, and the treatment plan requires continuous trial and error, which is time-consuming, laborious and inefficient.

Based on this, the embodiments of the present disclosure provide a method for training a deep reinforcement learning model for generating a treatment plan, a method for generating a treatment plan, a device, and a medium. The deep reinforcement learning model is configured to include a plurality of actor network layers and a critic network layer, and different actor network layers of the plurality of actor network layers are configured to output different types of target parameters included in the treatment plan. Thus, after acquiring the initial dose distribution state data of the objective target volume, the target data is determined based on the initial dose distribution state data of the objective target volume, current policy data of the plurality of actor network layers, and current policy data of the critic network layer. Then, the current policy data of the plurality of actor network layers and the current policy data of the critic network layer may be updated based on the target data, so as to complete the current training for the deep reinforcement learning model. The training process is iterated until a count of training the deep reinforcement learning model reaches a preset count, so as to obtain the deep reinforcement learning model that has been trained.

The target data includes: a final dose distribution of the objective target volume, multiple dose distribution state data of the objective target volume, a plurality of action sets output by the plurality of actor network layers corresponding to the multiple dose distribution state data respectively, a plurality of predicted values output by the critic network layer corresponding to the plurality of action sets respectively, a plurality of actual rewards corresponding to the plurality of action sets respectively, a predicted value of the objective target volume, and an actual reward of the objective target volume; where each action set of the plurality of action sets includes a target parameter combination composed of a plurality of different types of target parameters.

As set forth above, an overall task of determining action sets for different types of target parameters may be divided into a plurality of subtasks for determining actions for different types of target parameters in the present disclosure. Each actor network layer of the plurality of actor network layers in the deep reinforcement learning model completes a subtask, that is, the complex overall task is divided into the plurality of subtasks for calculation. Therefore, the plurality of actor network layers may improve the efficiency of determining target parameters. Due to the fact that the deep reinforcement learning model conforms to the characteristics of the design for the treatment plan with Gamma knife, and a processor in a computer system is able to repeat the trial and error process, target parameters with better performance may be automatically generated through the deep reinforcement learning model, resulting in generating a better treatment plan based on the target parameters with better performance. Thus, the dependence on clinical experience may be reduced, the effect of treatment plan may be improved without the need for manual setting of target parameters, and further, the efficiency for generating a treatment plan by a physician is improved.

The methods provided by the present disclosure mainly relate to a method for training a model and a method for generating a treatment plan. The application scenarios of the above two methods are introduced below.

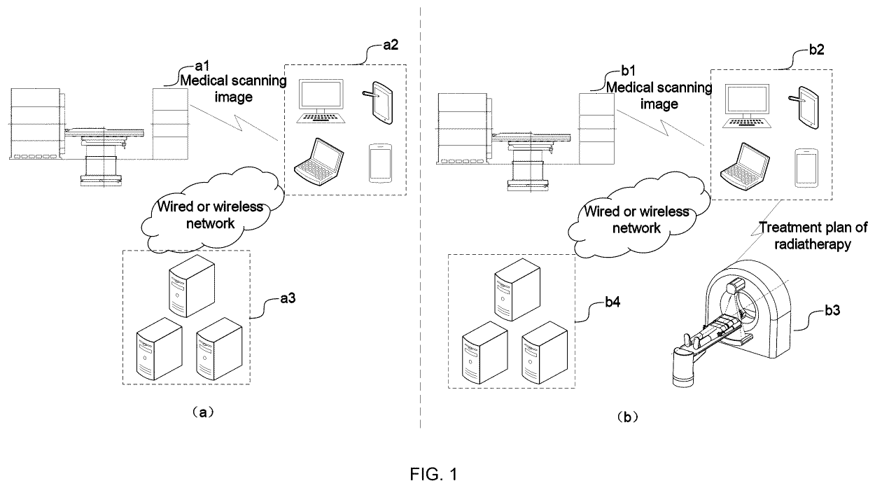

(a) in FIG. 1 is a schematic diagram of an exemplary application scenario of a method for training a model according to some embodiments. As shown in (a) in FIG. 1, an image scanning device a1 and an electronic device a2 are included in the application scenario.

The image scanning device a1 is a device configured to scan and display a tumor and surrounding normal tissues of a to-be-treated subject. In some embodiments, the imaging scanning device a1 may be at least one of the following devices: a computed tomography (CT) device, an emission computed tomography (ECT) device, a magnetic resonance imaging (MRI) device, a positron emission tomography (PET) device, or an ultrasound inspection device.

In some embodiments, the image scanning device a1 is configured to acquire a medical scanning image (e.g., image data corresponding to different target volumes respectively) of the to-be-treated subject and upload the medical scanning image to the electronic device a2. Thus, the electronic device a2 performs a method for training a model based on the medical scanning image of the to-be-treated subject.

The electronic device a2 is a device configured to train a deep reinforcement learning model. In some embodiments, the electronic device a2 may be at least one of the following devices: a smartphone, a smart watch, a desktop computer, a portable computer, a virtual reality terminal, an augmented reality terminal, a wireless terminal, or a laptop portable computer.

In some embodiments, the electronic device a2 may run a computer system that includes a processor for implementing the method for training the deep reinforcement learning model.

In some embodiments, a server a3 is also included in the application scenario. In some embodiments, the server a3 is configured to provide a background communication service for the image scanning device a1 and the electronic device a2.

The server a3 may be an independent physical server, a distributed file system or a server cluster composed of multiple physical servers, or at least one of cloud servers that can provide the following basic cloud computing services: providing cloud services, cloud databases, cloud computing, cloud functions, cloud storage, network services, cloud communication, middleware services, domain name services, security services, content distribution networks, mega data or an artificial intelligence platform, which may not be limited in the embodiments of the present disclosure. In some embodiments, a number of the above server a3 is one or more, which is not limited in the embodiments of the present disclosure. In some embodiments, the server a3 can also include other functions to provide more comprehensive and diversified services.

(b) in FIG. 1 is a schematic diagram of an exemplary application scenario of a method for generating a treatment plan according to some embodiments. As shown in (b) in FIG. 1, an image scanning device b1, an electronic device b2, and a radiotherapy device b3 are included in the application scenario.

A form of the image scanning device b1 is similar to that of the image scanning device a1 in (a) in FIG. 1, and more information on the image scanning device b1 may refer to the description on the image scanning device a1, which will not be repeated herein.

In the embodiments of the present disclosure, the image scanning device b1 may be configured to obtain a medical scanning image of the to-be-treated subject (e.g., image data of a to-be-treated target volume, etc.) and upload the medical scanning image of the to-be-treated subject to the electronic device b2. Thus, the electronic device b2 may execute the method for generating a treatment plan based on the medical scanning image.

The electronic device b2 is a device configured to generate a treatment plan by using the deep reinforcement learning model.

A form of the electronic device b2 is similar to that of the electronic device a2 in (a) in FIG. 1, and more information of the electronic device b2 may refer to the description of the electronic device a2, which will not be repeated herein.

In some embodiments, the electronic device b2 may run a computer system that includes a processor for implementing a method for generating a treatment plan using the deep reinforcement learning model.

The radiotherapy device b3 is a device used for radiotherapy. In some embodiments, the radiotherapy device b3 may be at least one of the following devices: a Gamma knife, a linear accelerator, a neutron knife, or an X-ray therapy machine.

In some embodiments, the radiotherapy device b3 is configured to receive a treatment plan from the electronic device b2, and perform radiotherapy on the to-be-treated subject based on the treatment plan.

In some embodiments, a server b4 is also included in the application scenario. In some embodiments, the server b4 is configured to provide a background communication service for the image scanning device b1, the electronic device b2, and the radiotherapy device b3 mentioned above.

A form of the server b4 is similar to that of the server a3 in (a) in FIG. 1, and more information of the server b4 may refer to the description of the server a3, which will not be repeated herein.

Based on the application scenario shown in (a) in FIG. 1, the method for training the model provided in the present disclosure would be introduced first.

The method for training a model provided in the embodiments of the present disclosure can be applied on the electronic device a2 in (a) in FIG. 1. The electronic device a2 can run a computer system including a processor for implementing the method for training a model.

It should be understood that since the method for training a deep reinforcement learning model involves a plurality of different processing and calculating processes, the processor can also implement the method by calling various suitable processing threads, which are not limited by the embodiments of the present disclosure. For the convenience of description, the processor is taken as an executing subject for illustration in the embodiments of the present disclosure.

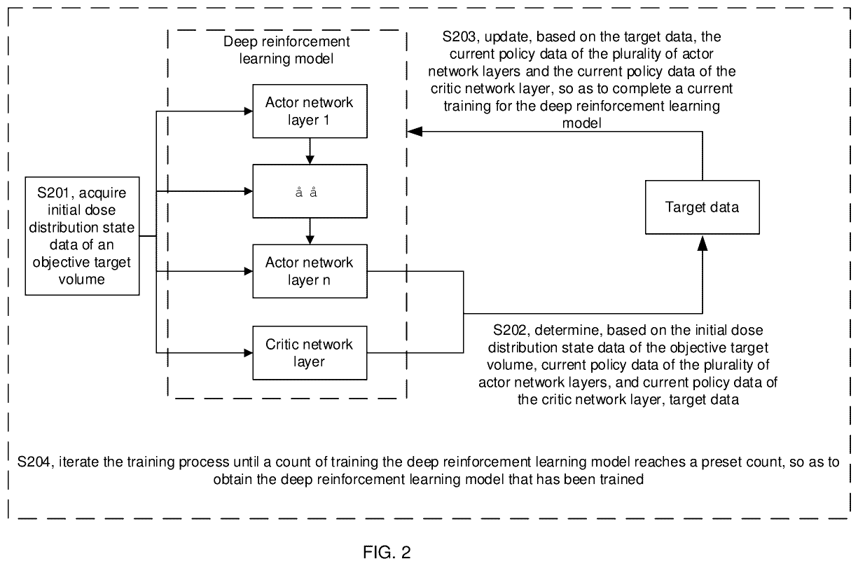

FIG. 2 is a flow chart of an exemplary method for training a deep reinforcement learning model for generating a treatment plan according to some embodiments. The deep reinforcement learning model is configured to include a plurality of actor network layers and a critic network layer, and different actor network layers of the plurality of actor network layers are configured to output different types of target parameters included in the treatment plan. As shown in FIG. 2, the method includes S201, S202, S203, and S204.

In S201, initial dose distribution state data of an objective target volume is acquired.

The objective target volume includes a to-be-treated area of a to-be-treated subject.

In some embodiments, the objective target volume may also be referred to as an objective planning target volume (PTV).

For example, the to-be-treated subject may be a phantom, a human body, or an animal. The objective target volume may be, for example, a tumor of the to-be-treated subject.

For example, in combination with (a) in FIG. 1, the image scanning device a1 may acquire image data of the to-be-treated subject and transmit the image data to the electronic device a2.

In some embodiments, the electronic device a2, after receiving the image data, may outline the image data to obtain contours (i.e., contour data) of different target volumes of the to-be-treated subject in the image data.

For example, the contour data may be a contour of a tumor.

In some embodiments, the electronic device a2 may also be connected to a third-party software program. This third-party software program may be configured to outline the image data to obtain the contour data.

In some embodiments, when the contour of the image data is outlined, the outlining operation may be performed by a physician on the electronic device a2. The electronic device a2 may respond to the outlining operation performed by the physician to obtain the contour data.

In some embodiments, the electronic device a2 may also automatically outline the contours of different target volumes of the to-be-treated subject in the image data through an outlining software.

After the image data and the contour data of the objective target volume are determined, the processor in the electronic device a2 may determine the initial dose distribution state data of the objective target volume based on the image data and the contour data of the objective target volume.

The initial dose distribution state data refers to dose distribution state data of the objective target volume when no target is placed.

The dose distribution state data includes: mask data of the objective target volume, a dose distribution of the objective target volume, a volume of an area in the objective target volume with an insufficient dose distribution, and a volume of an area in the objective target volume with an overflow dose.

Due to the fact that the initial dose distribution state data refer to the dose distribution state data of the objective target volume when no target is placed, dose distribution, a volume of an area in the objective target volume with an insufficient dose distribution, and a volume of an area in the objective target volume with an overflow dose in the initial dose distribution state data are all 0. In this case, the processor in the electronic device a2 can determine the initial dose distribution state data of the objective target volume only by determining the mask data of the objective target volume according to the image data and the contour data of the objective target volume.

Specifically, the image data of the objective target volume may include image data of the objective target volume and image data of an organ at risk (OAR).

The OAR refers to a normal organ around the objective target volume, i.e., an organ that has not undergone pathological changes.

The processor in electronic device a2 may process the image data and the contour data of the objective target volume to produce the mask data of the objective target volume. In a process of producing the mask data, the processor in the electronic device a2 may perform different operations on areas corresponding to the objective target volume and OARs based on the contour data, respectively, thereby distinguishing the areas corresponding to the objective target volume and the OARs.

At the same time, searching space for the positions of targets (i.e., positions for placing the targets) may be limited within the area corresponding to the objective target volume, thus avoiding the positions of objective target falling into the areas corresponding to OARs or areas corresponding to other tissues, thereby avoiding treatment damage to the areas corresponding to OARs or the areas corresponding to other tissues. Moreover, limiting the searching space for the positions of the targets within the areas corresponding to the objective target volume may reduce searching space for a specific shape in the objective target volume, thus improving the speed of training the model and generating the treatment plan.

For example, the processor in the electronic device a2 may construct a three-dimensional matrix with a uniform size. Image parameters of the areas corresponding to the objective target volume may be set as 1, image parameters of the areas corresponding to the OARs may be set as (−1), and image parameters of the areas corresponding to other tissues may be set as 0. Thus, mask data where the areas corresponding to the objective target volume and the areas corresponding to OARs are distinguished may be obtained.

In S202, target data is determined based on the initial dose distribution state data of the objective target volume, current policy data of the plurality of actor network layers, and current policy data of the critic network layer.

The plurality of actor network layers are deployed from top to bottom, that is, the plurality of actor network layers are connected in sequence. For example, as shown in FIG. 2, the plurality of actor network layers may include an actor network layer 1, . . . , and an actor network layer n, which are deployed from top to bottom; where n is an integer greater than or equal to 2.

In the practical application, as a target parameter typically include a size of a target, a weight of a target, and a position of a target, the plurality of actor network layers typically include at least two of a size actor network layer of a target, a position actor network layer of a target, or a weight actor network layer of a target. In some embodiments, when the target parameter further includes other types of parameters, a type and a number of actor network layers included in the corresponding plurality of actor network layers may also be adaptively adjusted based on the actual type and number of target parameters.

In some embodiments, the target data includes: multiple dose distribution state data of the objective target volume, a plurality of action sets output by the plurality of actor network layers corresponding to the multiple dose distribution state data respectively, a plurality of predicted values output by the critic network layer corresponding to the plurality of action sets respectively, a plurality of actual rewards corresponding to the plurality of action sets respectively, a predicted value of the objective target volume, and an actual reward of the objective target volume; where each action set of the plurality of action sets includes a target parameter combination composed of a plurality of different types of target parameters.

Due to the fact that a generation process of a treatment plan with Gamma knife is a process for placing targets in sequence, a dose distribution state of the objective target volume would change after the placement of a target is completed. Therefore, during the generation process of a complete treatment plan for the objective target volume, the dose distribution state of the objective target volume would change with the placement of one target after another.

Therefore, in the present disclosure, the target data can merely be obtained when a complete treatment plan for the objective target volume is generated.

Specifically, the processor in the electronic device a2, after obtaining the initial dose distribution state data, may input the initial dose distribution state data into each actor network layer of the plurality of actor network layers. Meanwhile, due to that the plurality of actor network layers are deployed from top to bottom, the processor in the electronic device a2 may also input the output data of an upper actor network layer into a lower actor network layer.

Different actor network layers of the plurality of actor network layers may output different types of target parameters selected for a first target based on corresponding initial policy data and initial dose distribution state data of the objective target volume. In this way, a first action set may be obtained based on output data of the plurality of actor network layers.

As the deep reinforcement learning model further includes a critic network layer, the processor in the electronic device a2 may simultaneously input the initial dose distribution state data into the critic network layer after the initial dose distribution state data is obtained. Therefore, while the first action set is determined, the critic network layer may determine a predicted value corresponding to the first action set based on initial policy data of the critic network layer and the initial dose distribution state data of the objective target volume.

After the first target is placed, the dose distribution state of the objective target volume changes. At this time, the processor in the electronic device a2 may also determine a dose distribution of the objective target volume based on the first action set, and determine an actual reward corresponding to the first action set based on the dose distribution of the objective target volume.

Then, the processor in the electronic device a2 may update the dose distribution state data of the objective target volume based on the dose distribution of the objective target volume, and repeat the above processes for placing a target based on the updated dose distribution state data of the objective target volume until a complete treatment plan of the objective target volume is generated. Then, a final dose distribution of the objective target volume, multiple dose distribution state data of the objective target volume, a plurality of action sets output by the plurality of actor network layers corresponding to the multiple dose distribution state data respectively, a plurality of predicted values output by the critic network layer corresponding to the plurality of action sets respectively, a plurality of actual rewards corresponding to the plurality of action sets respectively, a predicted value of the objective target volume, and an actual reward of the objective target volume are also obtained. In this way, the processor in the electronic device a2 may obtain the target data.

In S203, the current policy data of the plurality of actor network layers and the current policy data of the critic network layer are updated based on the target data, so as to complete a current training for the deep reinforcement learning model.

Specifically, after the target data is obtained, it means that the treatment plan for the objective target volume has been completed. In this case, the processor in the electronic device a2 may update the current policy data of the plurality of actor network layers and the current policy data of the critic network layer based on the target data.

It should be noted that the training process of the deep reinforcement learning model is equivalent to a process of trial and error for generating a treatment plan. Therefore, there are situations where the target data obtained by the plurality of actor network layers and the critic network layer is discarded for the reason that an update condition is not met. Therefore, in the embodiments of the present disclosure, the completion of a training for a deep reinforcement learning model does not mean that the current policy data of the plurality of actor network layers and the current policy data of the critic network layer would be updated.

In S204, a trained deep reinforcement learning model is obtained by iterating the training processes (i.e., the operations S201-S203) until a count of training the deep reinforcement learning model reaches a preset count.

Specifically, the training processes S201-S203 of the deep reinforcement learning model mentioned above are iterated until the count of iterating the training processes of the deep reinforcement learning model reaches the preset count, and thus, the trained deep reinforcement learning model is obtained.

In some embodiments, a buffer memory may be included in an electronic device. The buffer memory is configured to store the final dose distribution of the objective target volume, the multiple dose distribution state data of the objective target volume, a plurality of action sets output by the plurality of actor network layers corresponding to the multiple dose distribution state data respectively, the plurality of predicted values output by the critic network layer corresponding to the plurality of action sets respectively, the plurality of actual rewards corresponding to the plurality of action sets respectively, the predicted value of the objective target volume, and the actual reward of the objective target volume (i.e., the target data) in each stage during the process of updating the current policy data. Subsequently, through a correspondence stored in the buffer memory, various required data determined in each training may be found, and further, training is performed.

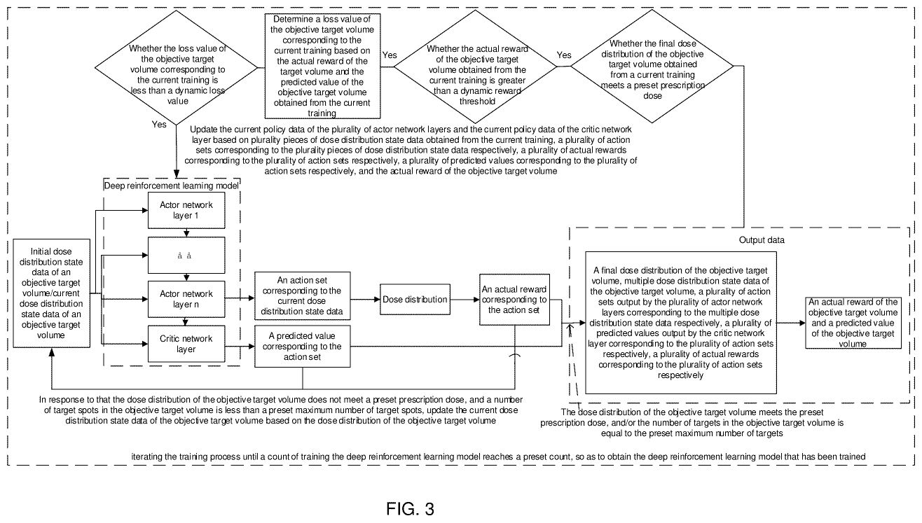

The above-mentioned methods for training a model in provided in some embodiments of the present disclosure are described in combination with FIG. 3. FIG. 3 illustrates an embodiment of a method for training a model according to some embodiments. As shown in FIG. 3, the method for training a model specifically includes the following steps.

In S1, initial dose distribution state data of an objective target volume is obtained.

The specific implementation process of this step may refer to the description of S201, which will not be repeated herein.

In S2, an action set corresponding to the current dose distribution state data output by the plurality of actor network layers is determined based on the current dose distribution state data of the objective target volume and current policy data of each actor network layer, and a predicted value output by the critic network layer corresponding to the action set is determined based on the current dose distribution state data of the objective target volume and the current policy data of the critic network layer.

Each actor network layer and the critic network layer may be provided with a piece of initial policy data in advance.

For an actor network layer, the policy data refers to a selection policy for a target parameter (also referred to as an action selection policy) when placing a target in the objective target volume, such as a policy of selecting a size of a target when placing the target.

For the critic network layer, the policy data is a prediction of a value of an action selection in a certain dose distribution state of the objective target volume. The policy data is used to determine a policy of the predicted value corresponding to a result of an action selection.

Due to the fact that a generation process of a treatment plan with Gamma knife is a process for placing targets in sequence, a dose distribution state of the objective target volume would change after the placement of a target is completed. Therefore, during the generation process of a complete treatment plan for the objective target volume, the current dose distribution state of the objective target volume would change with the placement of one target after another.

Therefore, after obtaining the current dose distribution state data of the objective target volume (for a first target, the current dose distribution state data of the objective target volume is the initial dose distribution state data of the objective target volume), the processor in the electronic device may input the current dose distribution state data of the objective target volume into each actor network layer of the plurality of actor network layers. A first actor network layer may determine a first action based on initial policy data of the first actor network layer and the current dose distribution state data of the objective target volume. The first action may represent a target parameter with a first type when placing the current target, such as a size of the target when placing the current target.

Then, the processor in the electronic device may input the output data of the first actor network layer (i.e., the first action) into the second actor network layer. The second actor network layer may determine a second action based on initial policy data of the second actor network layer, the current dose distribution state data of the objective target volume, and the output data of the first actor network layer (i.e., the first action). The second action may represent a target parameter with a second type when placing the current target, such as a position of the target when placing the current target.

Similarly, the processor in the electronic device may obtain an action set composed of a plurality of actions corresponding to the current dose distribution state data. The action set includes a plurality of different types of target parameters selected for the current target when placing the current target, such as the size, the position, and the weight of the target selected for the current target when placing the current target.

As the deep reinforcement learning model further includes the critic network layer, while determining the action set corresponding to the current dose distribution state data output by the plurality of actor network layers, the processor in the electronic device may input the initial dose distribution state data into the critic network layer after the current dose distribution state data of the objective target volume is obtained. The critic network layer may determine the predicted value corresponding to the above action set based on the initial policy data of the critic network layer and the current dose distribution state data of the objective target volume.

In S3, a dose distribution of the objective target volume is determined based on the action set corresponding to the current dose distribution state data, and an actual reward corresponding to the action set is determined based on the dose distribution of the objective target volume.

Specifically, after determining the action set corresponding to the current dose distribution state data, the processor in the electronic device may place a target in the objective target volume based on the action set. After placing the target, the dose distribution of the objective target volume would change. In this case, the processor in the electronic device may determine the changed dose distribution of the objective target volume.

In some embodiments, when the action set only includes two types of target parameters, for example, when the action set only includes a size of the target and a weight of the target, it is necessary to determine a position of the target to place the current target. In this case, the processor in the electronic device may call a shape-matching algorithm to determine the position of the target.

After all target parameters of the target (i.e., the size of the target, the position of the target, and the weight of the target) are obtained, the processor in the electronic device may determine the dose distribution of the objective target volume based on the target parameters.

It can be understood that when a number of the plurality of actor network layers is two, the deep reinforcement learning model may only generate two types of target parameters. When the types of target parameters exceed two, the determination of other types of target parameters may be carried out by calling corresponding algorithms through the processor in the electronic device.

Secondly, in order to obtain a trained deep reinforcement learning model in the subsequent training, the processor in the electronic device may also determine the actual reward corresponding to the action set based on the dose distribution of the objective target volume.

Specifically, since the dose distribution of the objective target volume is determined after the target is placed, the dose distribution of the objective target volume may truly reflect the contribution of the action set to the dose distribution of the objective target volume. In this way, after the action set is obtained, the processor in the electronic device may determine the actual reward corresponding to the action set based on the dose distribution of the objective target volume.

For example, the actual reward corresponding to the action set includes a positive reward and a negative reward. Compared the situation before placing the target corresponding to the action set with the situation after placing the target corresponding to the action set, a value obtained by summing a value obtained by multiplying a growth value of the conformity index of the objective target volume by a corresponding weight with a value obtained by multiplying a growth value of a dose coverage rate of the objective target volume by a corresponding weight may be designated as a positive reward. Compared the situation before placing the target corresponding to the action set with the situation after placing the target corresponding to the action set, a value obtained by multiplying a growth value of a dose overflow rate of the objective target volume by a corresponding weight may be designated as a negative reward.

In S4, in response to that the dose distribution of the objective target volume does not meet a preset prescription dose, and a number of targets in the objective target volume is less than a preset maximum number of targets, the current dose distribution state data of the objective target volume is updated based on the dose distribution of the objective target volume.

Specifically, after determining the dose distribution of the objective target volume, whether the dose distribution of the objective target volume meets the preset prescription dose, and/or whether the number of targets in the objective target volume is less than the preset maximum number of targets may be determined. When the dose distribution of the objective target volume does not meet the preset prescription dose, and a number of targets in the objective target volume is less than the preset maximum number of targets, it means that the treatment plan has not been completed in the objective target volume. In this case, the processor in the electronic device may update the current dose distribution state data of the objective target volume based on the dose distribution of the objective target volume. That is, the processor in the electronic device updates mask data of the objective target volume, the dose distribution of the objective target volume, a volume of an area in the objective target volume with an insufficient dose distribution, and a volume of an area in the objective target volume with an overflow dose.

In some embodiments, when the processor in the electronic device updates the current dose distribution state data of the objective target volume based on the dose distribution, the updated current dose distribution state data is obtained by performing a feature extraction on the dose distribution of the objective target volume.

Then, the processor in the electronic device may repeatedly execute the S2-S4 based on the updated dose distribution state data, select the corresponding action set for the subsequent targets, and determine the predicted reward value and the actual reward corresponding to the action set.

In S5, in response to that the dose distribution of the objective target volume meets the preset prescription dose, and/or the number of targets in the objective target volume is equal to the preset maximum number of targets, the final dose distribution of the objective target volume, the multiple dose distribution state data of the objective target volume, the plurality of action sets output by the plurality of actor network layers corresponding to the multiple dose distribution state data respectively, the plurality of predicted values output by the critic network layer corresponding to the plurality of action sets respectively, and the plurality of actual rewards corresponding to the plurality of action sets respectively are determined.

Specifically, when the dose distribution of the objective target volume meets the preset prescription dose, and/or a number of targets in the objective target volume is equal to the preset maximum number of targets, it means that the treatment plan has been completed in the objective target volume. In this case, a plurality of targets that are used to form the treatment plan have already been placed in the objective target volume. Correspondingly, the final dose distribution of the objective target volume, the multiple dose distribution state data of the objective target volume, the plurality of action sets output by the plurality of actor network layers corresponding to the multiple dose distribution state data respectively, the plurality of predicted values output by the critic network layer corresponding to the plurality of action sets respectively, and the plurality of actual rewards corresponding to the plurality of action sets respectively may be determined.

In S6, the actual reward of the objective target volume and the predicted value of the objective target volume are determined based on the final dose distribution of the objective target volume and the plurality of predicted values corresponding to the plurality of action sets respectively.

Specifically, when the dose distribution of the objective target volume meets the preset prescription dose, and/or a number of targets in the objective target volume is equal to the preset maximum number of targets, the final dose distribution of the objective target volume, and the plurality of predicted values corresponding to the plurality of action sets respectively may be obtained. In this case, the processor in the electronic device may determine the actual reward of the objective target volume based on the final dose distribution, and determine the predicted value of the objective target volume based on the plurality of predicted values corresponding to the plurality of action sets respectively.

In some embodiments, after the final dose distribution of the objective target volume is determined, the conformity index of the objective target volume and the dose coverage rate of the objective target volume may be determined based on the final dose distribution of the objective target volume, and the actual reward of the objective target volume may be determined based on the dose coverage rate and the conformity index of the objective target volume.

In some embodiments, after the plurality of predicted values corresponding to the plurality of action sets respectively are determined, the plurality of predicted values corresponding to the plurality of action sets respectively may be summed to obtain the predicted value of the objective target volume.

In some embodiments, the above method of updating, based on the target data, the current policy data of the plurality of actor network layers and the current policy data of the critic network layer, so as to complete a current training for the plurality of actor network layers and the critic network layer specifically includes the following step.

In S7, in response to that the final dose distribution of the objective target volume obtained from the current training meets a preset prescription dose, whether the actual reward of the objective target volume obtained from the current training is greater than a dynamic reward threshold is determined.

For example, the final dose distribution of the objective target volume is a dose distribution corresponding to the treatment plan of the objective target volume after completing the generation of the treatment plan for the objective target volume composed of the plurality of action sets.

It should be noted that since the treatment plan for the objective target volume is completed when the dose distribution of the objective target volume meets the preset prescription dose, and/or a number of targets in the objective target volume is equal to the preset maximum number of targets, therefore, if the treatment plan for the objective target volume is obtained in response that the dose distribution of the objective target volume meets the preset prescription dose, the processor in the electronic device may not need to determine whether the dose distribution corresponding to the treatment plan meets the preset prescription dose, and can proceed to the subsequent judgment steps. In some embodiments, the processor in the electronic device may also re-determine whether the dose distribution corresponding to the treatment plan meets the preset prescription dose.

However, if the treatment plan for the objective target volume is obtained when a number of targets in the objective target volume is equal to the preset maximum number of targets, since the treatment plan only meets the preset maximum number of targets, but does not necessarily meet the preset prescription dose, the processor in the electronic device needs to determine whether the dose distribution corresponding to the treatment plan meets the preset prescription dose.

If the dose distribution corresponding to the treatment plan does not meet the preset prescription dose, it means that the treatment plan is not a good treatment plan. Therefore, the processor in the electronic device may discard the target data corresponding to the treatment plan. That is, there is no need to update the current policy data of the plurality of actor network layers and the current policy data of the critic network layer based on the target data corresponding to the treatment plan.

Correspondingly, if the dose distribution corresponding to the treatment plan meets the preset prescription dose, it means that the treatment plan may meet the requirement for meeting the preset prescription dose. In this case, the processor in the electronic device may determine whether the actual reward of the objective target volume is greater than the dynamic reward threshold.

For example, the dynamic reward threshold is an actual reward of the objective target volume corresponding to target data used for updating the current policy data of the plurality of actor network layers and the current policy data of the critic network layer previously.

By determining whether the actual reward of the objective target volume obtained from the current training is greater than the dynamic reward threshold, whether the quality of the treatment plan is better than the quality of the treatment plan used for updating the current policy data of the plurality of actor network layers and the current policy data of the critic network layer previously may be determined.

When the actual reward of the objective target volume obtained from the current training is less than or equal to the dynamic reward threshold, the quality of the treatment plan is inferior to or equal to the quality of the treatment plan used for updating the current policy data of the plurality of actor network layers and the current policy data of the critic network layer previously. Therefore, the processor in the electronic device may discard the target data corresponding to the treatment plan. That is, there is no need to update the current policy data of the plurality of actor network layers and the current policy data of the critic network layer based on the target data corresponding to the treatment plan.

Correspondingly, when the actual reward of the objective target volume obtained from the current training is greater than the dynamic reward threshold, it means that the quality of the treatment plan is better than the quality of the treatment plan used for updating the current policy data of the plurality of actor network layers and the current policy data of the critic network layer previously. That is, the quality of the treatment plan is better. In this case, the electronic device may execute S8.

In S8, in response to that the actual reward of the objective target volume obtained from the current training is greater than the dynamic reward threshold, a loss value of the objective target volume corresponding to the current training is determined based on the actual reward and the predicted value of the objective target volume obtained from the current training.

Specifically, when the actual reward of the objective target volume obtained from the current training is greater than the dynamic reward threshold, it means that the quality of the treatment plan is better. In this case, the processor in the electronic device may determine the loss value of the objective target volume corresponding to the current training based on the actual reward of the objective target volume and the predicted value of the objective target volume obtained from the current training.

In some embodiments, the processor in the electronic device may use a preset loss function to determine the loss value of the objective target volume corresponding to the current training based on the actual reward of the objective target volume and the predicted value of the objective target volume obtained from the current training.

In some embodiments, the preset loss function may be a relative advantage function or another general loss function, which is not limited in the present disclosure.

Then, the processor in the electronic device may determine whether the loss value of the objective target volume corresponding to the current training is less than a dynamic loss value.

The dynamic loss value is a loss value of the objective target volume corresponding to target data used for updating the current policy data of the plurality of actor network layers and the current policy data of the critic network layer previously.

If the loss value of the objective target volume corresponding to the current training is greater than or equal to the loss value of the objective target volume corresponding to target data used for updating the current policy data of the plurality of actor network layers and the current policy data of the critic network layer previously, it means that the deep reinforcement learning model is inaccurate in predicting the quality of the treatment plan (i.e., a prediction for a contribution value of each selection of an action set to the treatment plan). Therefore, the processor in the electronic device may discard the target data corresponding to the treatment plan. That is, there is no need to update the current policy data of the plurality of actor network layers and the current policy data of the critic network layer based on the target data corresponding to the treatment plan.

If the loss value of the objective target volume corresponding to the current training is less than the loss value of the objective target volume corresponding to target data used for updating the current policy data of the plurality of actor network layers and the current policy data of the critic network layer previously, it means that the deep reinforcement learning model is accurate in predicting the quality of the treatment plan (i.e., a prediction for a contribution value of each selection of an action set to the treatment plan). In this case, the processor in the electronic device may execute S9.

In S9, in response to that the loss value of the objective target volume corresponding to the current training is less than a dynamic loss value, the current policy data of the plurality of actor network layers and the current policy data of the critic network layer are updated based on the multiple dose distribution state data obtained from the current training, the plurality of action sets corresponding to the multiple dose distribution state data respectively, the plurality of predicted values corresponding to the plurality of action sets respectively, and the actual reward of the objective target volume.

In some embodiments, the above method of updating the current policy data of the plurality of actor network layers and the current policy data of the critic network layer based on the multiple dose distribution state data obtained from the current training, the plurality of action sets corresponding to the multiple dose distribution state data respectively, the plurality of predicted values corresponding to the plurality of action sets respectively, and the actual reward of the objective target volume specifically includes: determining an actual cumulated reward value of the plurality of action sets based on the plurality of actual rewards corresponding to the plurality of action sets respectively and the actual reward of the objective target volume, and updating the current policy data of the plurality of actor network layers based on the multiple dose distribution state data, the plurality of action sets corresponding to the multiple dose distribution state data respectively, and the actual cumulated reward value of the plurality of action sets, and updating the current policy data of the critic network layer based on the multiple dose distribution state data, the plurality of action sets corresponding to the multiple dose distribution state data respectively, and the plurality of predicted values corresponding to the plurality of action sets respectively.

Specifically, the quality of the treatment plan for the objective target volume is a result of the cooperation of the plurality of action sets. Therefore, under a certain dose distribution state of the objective target volume, an actual reward of a selected action set being high does not mean that the quality of the treatment plan for the objective target volume is good. If the quality of the treatment plan for the objective target volume is good, a reference value for the selection of the action set may be great. If the quality of the treatment plan for the objective target volume is not good, the reference value of the selection for the action set may be reduced. Therefore, after obtaining the plurality of action sets corresponding to the multiple dose distribution state data respectively, the actual cumulated reward value of the plurality of action sets may also be determined based on the plurality of actual rewards corresponding to the plurality of action sets respectively and the actual reward of the objective target volume.

The actual cumulated reward value may include an actual reward of each action set and a delayed reward value after the completion of the entire treatment plan. The delayed reward value is determined based on the actual reward of the objective target volume. That is, after the treatment plan for the objective target volume is performed completely, the actual reward of the objective target volume is determined based on the dose distribution of the treatment plan, and the actual reward is distributed to each action set that constitutes the treatment plan based on a weight, thus forming a delayed reward value of the action set. The actual reward and the delayed reward value of each action set are accumulated to obtain the actual cumulated reward value of each action set.

When the actual cumulated reward value is high, it may be considered that the selected action set under the dose distribution state has a high reference value. In this case, the current policy data of the plurality of actor network layers may be updated based on the multiple dose distribution state data, the plurality of action sets corresponding to the multiple dose distribution state data respectively, and the actual cumulated reward values of the plurality of action sets.

Meanwhile, the loss value of the objective target volume is obtained based on the predicted value of the objective target volume, and the predicted value of the objective target volume is an accumulation of predicted values of the plurality of action sets, thus when the loss value of the objective target volume is less than the dynamic loss value, it means that the predicted values of the plurality of action sets may have a high reference value. In this case, the current policy data of the critic network layer can be updated based on the multiple dose distribution state data, the plurality of action sets corresponding to the multiple dose distribution state data respectively, and the plurality of predicted values corresponding to the plurality of action sets respectively.

In S10, a trained deep reinforcement learning model is obtained by iterating the training processes (i.e., the operations S1-S9) until a count of training the deep reinforcement learning model reaches a preset count.

The specific implementation process of this step may refer to the description of S204, which will not be repeated herein.

In some embodiments, the plurality of actor network layers include at least two of a size actor network layer of a target, a position actor network layer of a target, or a weight actor network layer of a target. When the plurality of actor network layers include a first actor network layer and a second actor network layer deployed from top to bottom, as described in S1 above, determining an action set output by the plurality of actor network layers corresponding to the current dose distribution state data based on the current dose distribution state data of the objective target volume, and the current policy data of each actor network layer includes:

determining, based on the current dose distribution state data and current policy data of the first actor network layer, a first action; and determining, based on the current dose distribution state data, the first action, and current policy data of the second actor network layer, a second action corresponding to the first action.

In some embodiments, the first actor network layer and the second actor network layer may be any two different actor network layers used for selecting a size of a target, an actor network layer used for selecting a position of a target, and an actor network layer used for selecting a weight of a target.

Correspondingly, the first action and the second action mentioned above may be any two actions with different types, of an action for selecting a size of a target, an action for selecting a position of a target, and an action for selecting a weight of a target when a target is placed under the current dose distribution state data.

It should be noted that the first actor network layer corresponds to the first action, and the second actor network layer also corresponds to the second action. For example, when the first actor network layer is an actor network layer for selecting a size of a target, the first action is an action for selecting a size of a target.

For example, assuming that the first actor network layer is an actor network layer for selecting a size of a target, and the second actor network layer is an actor network layer for selecting a weight of a target, the first action is an action for selecting a size of a target, and the second action is an action for selecting a weight of a target.

When a target is placed under the current dose distribution state data, the processor in the electronic device may input the current dose distribution state data into the first actor network layer for selecting a size of the target. The first actor network layer may determine the size of the target when placing the target under the current dose distribution state data based on the current policy data of the first actor network layer, that is, determine the size of the target when placing the target under the current dose distribution state data.

In some embodiments, after the size of the target is determined when placing the target under the current dose distribution state data, the processor in the electronic device may also call a shape-matching algorithm to determine a position of the target corresponding to the size of the target within the objective target volume based on the size of the target when placing the target under the current dose distribution state data.

Then, the processor in the electronic device may input the current dose distribution state data and the action of selecting the size of the target when placing the target under the current dose distribution state data into the second actor network layer for selecting the weight of the target. The second actor network layer may determine the weight of the target when placing the target under the current dose distribution state data based on the current policy data of the second actor network layer, that is determine the weight of the target when placing the target under the current dose distribution state data.

In this way, the processor in the electronic device may obtain the action set composed of the first action and the second action.

It can be understood that when a number of the plurality of actor network layers is two, the deep reinforcement learning model may only generate two types of target parameters. When the types of target parameters exceed two, the determination of other types of target parameters may be carried out by calling corresponding algorithms through the processor in the electronic device.

In still some embodiments, when the plurality of actor network layers include the first actor network layer, the second actor network layer, and a third actor network layer deployed from top to bottom, as described in S1 above, determining an action set output by the plurality of actor network layers corresponding to the current dose distribution state data based on the current dose distribution state data of the objective target volume, and the current policy data of each actor network layer includes:

determining, based on the current dose distribution state data and current policy data of the first actor network layer, a first action; determining, based on the current dose distribution state data, the first action, and current policy data of the second actor network layer, a second action corresponding to the first action; and determining, based on the current dose distribution state data, the first section, the second section, and current policy data of the third actor network layer, a third action corresponding to the first action.

In some embodiments, the first actor network layer, the second actor network layer, and the third actor network layer may be different actor network layers in an actor network layer for selecting a size of a target, an actor network layer for selecting a position of a target, and an actor network layer for selecting a weight of a target.

Correspondingly, the first action, the second action, and the third action mentioned above may be different actions in an action for selecting a size of the target, an action for selecting a position of the target, and an action for selecting a weight of the target when placing the target under the current dose distribution state data. It should be noted that the first actor network layer corresponds to the first action, the second actor network layer corresponds to the second action, and the third actor network layer corresponds to the third action. For example, when the first actor network layer is an actor network layer for selecting a size of a target, the first action is an action for selecting a size of a target.

For example, assuming that the first actor network layer is an actor network layer for selecting a size of a target, the second actor network layer is an actor network layer for selecting a position of a target, and the third actor network layer is an actor network layer for selecting a weight of a target, then the first action is an action for selecting a size of a target, and the second action is an action for selecting a position of a target, and the third action is an action for selecting a weight of a target.

When a target is placed under the current dose distribution state data, the processor in the electronic device may input the current dose distribution state data into the first actor network layer for selecting a size of the target. The first actor network layer may determine the size of the target when placing the target under the current dose distribution state data based on the current policy data of the first actor network layer, that is, determine the size of the target when placing the target under the current dose distribution state data.

Then, the processor in the electronic device may input the current dose distribution state data and the action of selecting the size of the target when placing the target under the current dose distribution state data into the second actor network layer for selecting the position of the target. The second actor network layer may determine the position of the target when placing the target under the current dose distribution state data based on the current policy data of the second actor network layer, that is determine the position of the target when placing the target under the current dose distribution state data.

Then, the processor in the electronic device may input the current dose distribution state data, the action for selecting the size of the target when placing the target under the current dose distribution state data, and the action for selecting the position of the target when placing the target under the current dose distribution state data into the third actor network layer for selecting a weight of the target. The third actor network layer may determine the weight of the target when placing the target under the current dose distribution state data based on the current policy data of the third actor network layer, that is determine the weight of the target when placing the target under the current dose distribution state data.

In this way, the processor in the electronic device may obtain an action set composed of the first action, second action, and third action.

The method for generating a treatment plan is introduced based on the electronic device as follows.

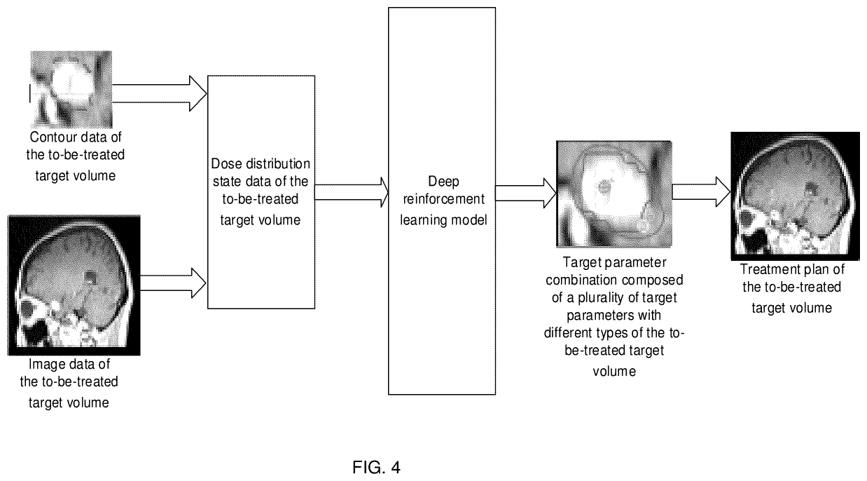

FIG. 4 is a flow chart of an exemplary method for generating a treatment plan executed by a processor according to some embodiments. As shown in FIG. 4, the method includes the following steps.

In S401, image data of a to-be-treated target volume and contour data of the to-be-treated target volume are acquired.

A specific process of acquiring the image data and the contour data of the to-be-treated objective target volume may refer to a specific process of acquiring the image data and the contour data of the different target volumes in S201, which will not be repeated herein.

For example, as shown in FIG. 4, the image data of the to-be-treated target volume may be a CT image of the head of the to-be-treated subject, and the contour data may be contour data of the to-be-treated target volume in the head of the to-be-treated subject.

In S402, dose distribution state data of the to-be-treated target volume is determined based on the image data and the contour data of the to-be-treated target volume.

A specific process of determining the dose distribution state data of the to-be-treated target volume may refer to specific process of determining the initial dose distribution state data of the to-be-treated target volume in S201, which will not be repeated herein.

In S403, the dose distribution state data of the to-be-treated target volume into a deep reinforcement learning model is inputted, so as to obtain a target parameter combination composed of a plurality of different types of target parameters of the to-be-treated target volume.

The deep reinforcement learning model is obtained based on the method for training the model described in FIG. 2 or FIG. 3.

For example, as shown in FIG. 4, the target parameter combination composed of a plurality of different types of target parameters of the to-be-treated target volume output by the deep reinforcement learning model may include a target parameter combination composed of at least two of a size of a target, a position of a target, and a weight of a target.

In S404, the treatment plan for the to-be-treated target volume is generated based on the target parameter combination.

It should be noted that when the above target parameter combination includes two types of target parameters (e.g., a size of a target, and a position of a target), the processor may determine the remaining target parameters that constitute the treatment plan by calling corresponding algorithms (e.g., calling a position matching algorithm to determine a position of a target based on the target parameter combination composed of a size of a target and a weight of a target) to generate the treatment plan.

Taking a target parameter combination being composed of a size of a target, a position of a target, and a weight of a target as an example, the process of generating a treatment plan by the processor is described as follows. It can be understood that the target parameter combination may also be at least two types of target parameters of a size of a target, a position of a target, and a weight of a target.

Firstly, the processor acquires image data of a to-be-treated target volume and contour data of the to-be-treated target volume. Then, based on the image data and the contour data of the to-be-treated target volume that have been acquired, the processor determines the dose distribution status data of the to-be-treated target volume, and inputs the dose distribution status data of the to-be-treated target volume into a deep reinforcement learning model trained using the method for training a model shown in FIG. 2 or FIG. 3, thus a size of a first target, a position of the first target, and a weight of the first target are output from the deep reinforcement learning model. That is, the relevant target parameters of the first target in the treatment plan have been determined.

Then, the processor calculates the dose distribution of the to-be-treated target volume based on the relevant target parameters of the first target, and determines the current dose distribution status data of the to-be-treated target volume based on the dose distribution of the to-be-treated target volume. The current dose distribution status data of the to-be-treated target volume is input into the deep reinforcement learning model to obtain a size, a position, and a weight of a second target output by the deep reinforcement learning model.

Iterating the processes of determining a size, a position, and a weight of a target mentioned above to determine relevant parameters of other targets until the dose distribution of the to-be-treated target volume meets the prescription dose, and/or a number of targets in the to-be-treated target volume is equal to a preset maximum number of targets, then the target parameter combination for the to-be-treated target volume is determined, and the treatment plan is determined based on the target parameter combination. The target parameter combination of the to-be-treated target volume includes related parameters of the plurality of targets. That is, the target parameter combination includes a plurality of sets ((S1, P1, W1), (S2, P2, W2), . . . , (Sn, Pn, Wn)) composed of a size(S) of a target, a position (P) of a target, and a weight (W) of a target.

It can be understood that a target weight of a target mentioned above may also be obtained without the deep reinforcement learning model, and may be defaulted to 1.

The treatment plan may be displayed in a displayed form of an image corresponding to image data of a to-be-treated subject. In the image corresponding to the image data of the to-be-treated subject, the target parameter combination may be clearly labeled.

In some embodiments, after determining the treatment plan for the to-be-treated target volume, a physician may also manually adjust the target parameter combination for the treatment plan for the to-be-treated target volume based on experience, and thus, the treatment plan for the to-be-treated target volume is determined based on the adjusted target parameter combination.

The above description mainly involves the methods provided in some embodiments of the present disclosure from the perspective of a computer system. In order to achieve the above functions, the computer system includes hardware structures and/or software modules configured to execute the functions. Those skilled persons in the art should easily realize that the embodiments of the present disclosure can be implemented in the form of a hardware or a combination of hardware and computer software in combination with the units and algorithm steps described in the embodiments of the present disclosure. Whether a certain function is executed by hardware or computer software depends on the specific application and design constraints of the technical solution. Professional technicians may use different methods to implement the described functions for each specific application, but such implementation should not be considered beyond the scope of the present disclosure.

The embodiments of the present disclosure may be divided into functional modules based on the computer system. For example, functional modules may be divided corresponding to different functions, or two or more functions may be integrated into one processing module. An integrated module mentioned above may be implemented in both hardware and software functional modules. In some embodiments, the modules in the embodiments of the present disclosure may be divided for illustration and may merely serve as a logical functional division. In the actual implementation, the modules in the present disclosure may be divided by other methods.

In some embodiments of the present disclosure, an electronic device is further provided. The electronic device includes at least one processor, and a memory connected in communication with the at least one processor. The memory stores instructions that can be executed by the at least one processor, and the instructions are executed by the at least one processor, such that the at least one processor can execute the method for generating the treatment plan provided by the present disclosure.

In some embodiments of the present disclosure, a non-transitory computer readable storage medium stored computer instructions thereon is further provided. The computer instructions are used to cause the electronic device to implement the method for generating a treatment plan.

In some embodiments of the present disclosure, a computer program product is further provided. The computer program product includes a computer program. The computer program, upon being executed by a processor, implements the method for training a deep reinforcement learning model for generating a treatment plan, and the method for generating a treatment plan.

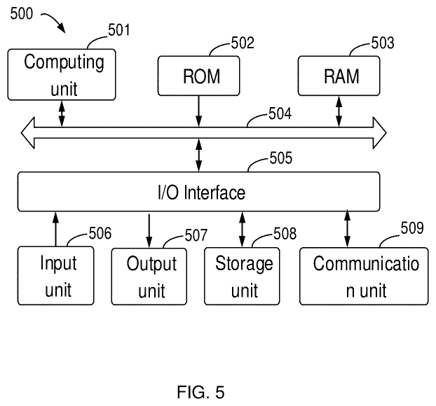

In some embodiments, the electronic device may be the electronic device a2 or the electronic device b2 shown in FIG. 1. FIG. 5 is a block diagram of an example electronic device 500 that can be configured to implement some embodiment of the present disclosure. The electronic device 500 may include, but is not limited to various forms of digital computers, such as a laptop, a desktop computer, a workstation, a personal digital assistant, a server, a blade server, a mainframe computer, or other suitable computers. The electronic device 500 may include, but is not limited to various forms of mobile devices, such as a personal digital assistant, a cellular phone, a smartphone, a wearable device, or other similar computing devices. The components, connections and relationships between the components herein, and functions of the components are merely illustrative, and are not intended to limit the scope of the present disclosure.

As shown in FIG. 5, the electronic device 500 may include a computing unit 501, which may perform various appropriate actions and processing based on computer programs stored in a read only memory (ROM) 502 or computer programs loaded from a storage unit 508 into a random access memory (RAM) 503. In RAM 503, various programs and data required for the operation of the electronic device 500 may also be stored. The computing unit 501, the ROM 502, and the RAM 503 are connected to each other through a bus 504. The input/output (I/O) interface 505 may also connected to the bus 504.

A plurality of components in the electronic device 500 may be connected to the I/O interface 505, including: an input unit 506 (e.g., a keyboard, a mouse, etc.), an output unit 507 (e.g., various types of displays, speakers, etc.), a storage unit 508 (e.g., a magnetic disk, an optical disc, etc.), and a communication unit 509 (e.g., a network card, a modem, a wireless communication transceiver, etc.). The communication unit 509 allows the electronic device 500 to exchange information/data with other devices through computer networks, such as the Internet and/or various telecommunications networks.

The computing unit 501 may be various general and/or specialized processing components with processing and computing capabilities. The computing unit 501 includes, but are not be limited to, a central processing unit (CPU), a graphics processing unit (GPU), various specialized artificial intelligence (AI) computing chips, various computing units for running machine learning model algorithms, a digital signal processor (DSP), and any appropriate processor, a controller, or a microcontroller, etc. The computing unit 501 executes various methods and processes described above, such as the method for generating a treatment plan. For example, in some embodiments, the method for generating a treatment plan may be implemented as a computer software program, which is tangibly included in a machine readable medium, such as the storage unit 508. In some embodiments, some or all of the computer programs may be loaded and/or installed on the electronic device 500 via the ROM 502 and/or the communication unit 509. When a computer program is loaded into the RAM 503 and executed by the computing unit 501, one or more steps of the method for generating a treatment plan described above may be executed. In other embodiments, the computing unit 501 may be configured to execute the method for generating a treatment plan by any other appropriate means (e.g., by virtue of firmware).

The various implementations of systems and technologies described above in the present disclosure could be implemented in a digital electronic circuit system, an integrated circuit system, a field programmable gate array (FPGA), an application specific integrated circuit (ASIC), an application specific standard part (ASSP), a system on chip (SOC) systems, a complex programmable logic device (CPLD), a computer hardware, firmware, software, and/or any combination thereof. These various implementation methods may include: implementation in one or more computer programs, which could be executed and/or interpreted on a programmable system including at least one programmable processor. The programmable processor may be a dedicated or general-purpose programmable processor that can receive data and instructions from a storage system, at least one input device, and at least one output device, and transmit data and instructions to the storage system, the at least one input device, and the at least one output device.

The program code for implementing the method described in the present disclosure may be written in any combination of one or more programming languages. These program codes may be provided to a processor or a controller of a general-purpose computer, a specialized computer, or other programmable data processing devices, so that the program code, when executed by the processor or the controller, enables the functions/operations specified in the flow chart and/or block diagram to be implemented. The program codes may be completely executed on the machine, partially executed on the machine, as an independent software package partially executed on the machine, and partially executed on remote machines or completely executed on remote machines or servers.

In the context of the present disclosure, a machine readable medium may be a tangible medium that can contain or store programs for use by or in combination with instruction execution systems, apparatuses, or devices. The machine readable medium may be machine readable signal medium or machine readable storage medium. The machine readable medium may include, but is not limited to an electronic, a magnetic, an optical, an electromagnetic, an infrared, or a semiconductor system, apparatus, or device, or any combination thereof. More specific examples of machine readable storage medium include an electrical connection based on one or more wires, a portable computer disk, a hard drive, a random access memory, a read-only memory (ROM), an erasable programmable read only memory (EPROM or flash memory), an optical fiber, a compact disc read only memory (CD-ROM), an optical storage device, a magnetic storage device, or any combination thereof.

In order to provide interaction with users, the system and technology described in the present disclosure can be implemented on a computer, which includes a display device (e.g., a cathode ray tube (CRT) or a liquid crystal display (LCD)) for displaying information to users. The computer may further include a keyboard and a pointing device (e.g., a mouse or trackball). Thus, the users can provide input to the computer via the keyboard and the pointing device. Other types of devices may also be used to provide interaction with users. For example, the feedback provided to users may be any form of sensory feedback (e.g., visual feedback, auditory feedback, or tactile feedback). Further, the input from the users can be received in any form (including voice input, voice input, or tactile input).

The system and technology described in the present disclosure may be implemented in a computing system that includes a background component (e.g., serving as a data server), or in a computing system that includes a middleware component (e.g., an application server), or in a computing system that includes a front-end component (e.g., a user computer with a graphical user interface or web browser through which users can interact with the implementation of the system and technology described in the present disclosure), or in a computing system that includes any combination of the background component, the middleware components, or the front-end component. The components of the system may be interconnected through any form or medium of digital data communication (e.g., a communication network). Examples of the communication network include: a local area network (LAN), a wide area network (WAN), or the Internet.

A computer system may include both clients and servers. The client and server are generally far away from each other and typically interact through the communication network. A client-server relationship is generated by running computer programs on corresponding computers that have client-server relationships with each other. The server may be a cloud server, a distributed system server, or a server that combines block chain.

It should be understood that various forms of processes shown in the present disclosure may be reordered, added, or deleted. For example, the steps described in the present disclosure may be executed in parallel, in sequence, or in different orders as long as the expected result of the technical solution related to the present disclosure can be achieved, which may not be limited in the present disclosure.

The specific implementations do not limit the scope of the present disclosure. Those skilled in the art should understand that various modifications, combinations, sub-combinations, and substitutions may be made based on design requirements and other factors. Any modifications, equivalent substitutions, and improvements made within the principles of the present disclosure shall be included within the scope of the present disclosure.

1. A method for training a deep reinforcement learning model for generating a treatment plan, wherein the deep reinforcement learning model is configured to include a plurality of actor network layers and a critic network layer, and different actor network layers of the plurality of actor network layers are configured to output different types of target parameters included in the treatment plan, wherein the method comprises:
performing a training process, the training process including the following operations:
acquiring initial dose distribution state data of an objective target volume;
determining, based on the initial dose distribution state data of the objective target volume, current policy data of the plurality of actor network layers, and current policy data of the critic network layer, target data; and
updating, based on the target data, the current policy data of the plurality of actor network layers and the current policy data of the critic network layer, so as to complete a current training for the deep reinforcement learning model; and
iterating the training process until a count of training the deep reinforcement learning model reaches a preset count, so as to obtain the deep reinforcement learning model that has been trained;
wherein the target data comprises: a final dose distribution of the objective target volume, multiple dose distribution state data of the objective target volume, a plurality of action sets output by the plurality of actor network layers corresponding to the multiple dose distribution state data respectively, a plurality of predicted values output by the critic network layer corresponding to the plurality of action sets respectively, a plurality of actual rewards corresponding to the plurality of action sets respectively, a predicted value of the objective target volume, and an actual reward of the objective target volume; wherein each action set of the plurality of action sets comprises a target parameter combination composed of a plurality of different types of target parameters.

2. The method according to claim 1, wherein the plurality of actor network layers comprise at least two of a size actor network layer of a target, a position actor network layer of a target, or a weight actor network layer of a target.

3. The method according to claim 2, wherein the determining, based on the initial dose distribution state data of the objective target volume, the current policy data of the plurality of actor network layers, and the current policy data of the critic network layer, the target data comprises:
determining, based on the current dose distribution state data of the objective target volume and current policy data of each actor network layer of the plurality of actor network layers, an action set corresponding to the current dose distribution state data output by the plurality of actor network layers;
determining, based on the current dose distribution state data of the objective target volume and the current policy data of the critic network layer, a predicted value output by the critic network layer corresponding to the action set;
determining, based on the action set corresponding to the current dose distribution state data, a dose distribution of the objective target volume, and determining an actual reward corresponding to the action set based on the dose distribution of the objective target volume;
in response to that the dose distribution of the objective target volume does not meet a preset prescription dose, and a number of targets in the objective target volume is less than a preset maximum number of targets, updating the current dose distribution state data of the objective target volume based on the dose distribution of the objective target volume; or, in response to that the dose distribution of the objective target volume meets the preset prescription dose, and/or the number of targets in the objective target volume is equal to the preset maximum number of targets, determining the final dose distribution of the objective target volume, the multiple dose distribution state data of the objective target volume, the plurality of action sets output by the plurality of actor network layers corresponding to the multiple dose distribution state data respectively, the plurality of predicted values output by the critic network layer corresponding to the plurality of action sets respectively, and the plurality of actual rewards corresponding to the plurality of action sets respectively; and
determining, based on the final dose distribution of the objective target volume and the plurality of predicted values corresponding to the plurality of action sets respectively, the predicted value of the objective target volume and the actual reward of the objective target volume.

4. The method according to claim 3, wherein when the plurality of actor network layers comprises a first actor network layer and a second actor network layer deployed from top to bottom, the determining, based on the current dose distribution state data of the objective target volume and the current policy data of each actor network layer of the plurality of actor network layers, the action set corresponding to the current dose distribution state data output by the plurality of actor network layers comprises:
determining, based on the current dose distribution state data and current policy data of the first actor network layer, a first action; and
determining, based on the current dose distribution state data, the first action, and current policy data of the second actor network layer, a second action corresponding to the first action.

5. The method according to claim 3, wherein when the plurality of actor network layers comprises a first actor network layer, a second actor network layer, and a third actor network layer deployed from top to bottom, the determining, based on the current dose distribution state data of the objective target volume and the current policy data of each actor network layer of the plurality of actor network layers, the action set corresponding to the current dose distribution state data output by the plurality of actor network layers comprises:
determining, based on the current dose distribution state data and current policy data of the first actor network layer, a first action;
determining, based on the current dose distribution state data, the first action, and current policy data of the second actor network layer, a second action corresponding to the first action; and
determining, based on the current dose distribution state data, the first action, the second action, and current policy data of the third actor network layer, a third action corresponding to the first action.

6. The method according to claim 1, wherein updating, based on the target data, the current policy data of the plurality of actor network layers and the current policy data of the critic network layer, so as to complete a current training for the deep reinforcement learning model comprises:
in response to that the final dose distribution of the objective target volume obtained from the current training meets a preset prescription dose, determining whether the actual reward of the objective target volume obtained from the current training is greater than a dynamic reward threshold; wherein the dynamic reward threshold is an actual reward of the objective target volume corresponding to target data used for updating the current policy data of the plurality of actor network layers and the current policy data of the critic network layer previously;
in response to that the actual reward of the objective target volume obtained from the current training is greater than the dynamic reward threshold, determining a loss value of the objective target volume corresponding to the current training based on the actual reward and the predicted value of the objective target volume obtained from the current training;
in response to that the loss value of the objective target volume corresponding to the current training is less than a dynamic loss value, updating the current policy data of the plurality of actor network layers and the current policy data of the critic network layer based on the multiple dose distribution state data obtained from the current training, the plurality of action sets corresponding to the multiple dose distribution state data respectively, the plurality of predicted values corresponding to the plurality of action sets respectively, and the actual reward of the objective target volume; wherein the dynamic loss value is a loss value of the objective target volume corresponding to target data used for updating the current policy data of the plurality of actor network layers and the current policy data of the critic network layer previously.

7. The method according to claim 6, wherein the updating the current policy data of the plurality of actor network layers and the current policy data of the critic network layer based on the multiple dose distribution state data obtained from the current training, the plurality of action sets corresponding to the multiple dose distribution state data respectively, the plurality of predicted values corresponding to the plurality of action sets respectively, and the actual reward of the objective target volume comprises:
determining, based on the plurality of actual rewards corresponding to the plurality of action sets and the actual reward of the objective target volume, an actual cumulated reward value of the plurality of action sets, and updating the current policy data of the plurality of actor network layers based on the multiple dose distribution state data, the plurality of action sets corresponding to the multiple dose distribution state data respectively, and the actual cumulated reward value of the plurality of action sets; and
updating the current policy data of the critic network layer based on the multiple dose distribution state data, the plurality of action sets corresponding to the multiple dose distribution state data respectively, and the plurality of predicted values corresponding to the plurality of action sets respectively.

8. A method for generating a treatment plan, comprising:
acquiring image data of a to-be-treated target volume and contour data of the to-be-treated target volume;
determining, based on the image data and the contour data, dose distribution state data of the to-be-treated target volume;
inputting the dose distribution state data of the to-be-treated target volume into a deep reinforcement learning model, so as to obtain a target parameter combination composed of a plurality of different types of target parameters of the to-be-treated target volume; wherein the deep reinforcement learning model is configured to include a plurality of actor network layers and a critic network layer, and different actor network layers of the plurality of actor network layers are configured to output different types of target parameters included in the treatment plan, wherein the deep reinforcement learning model is trained by the following operations:
performing a training process, the training process including the following operations:
acquiring initial dose distribution state data of an objective target volume;
determining, based on the initial dose distribution state data of the objective target volume, current policy data of the plurality of actor network layers, and current policy data of the critic network layer, target data; and
updating, based on the target data, the current policy data of the plurality of actor network layers and the current policy data of the critic network layer, so as to complete a current training for the deep reinforcement learning model; and
iterating the training process until a count of training the deep reinforcement learning model reaches a preset count, so as to obtain the deep reinforcement learning model that has been trained;
wherein the target data comprises: a final dose distribution of the objective target volume, multiple dose distribution state data of the objective target volume, a plurality of action sets output by the plurality of actor network layers corresponding to the multiple dose distribution state data respectively, a plurality of predicted values output by the critic network layer corresponding to the plurality of action sets respectively, a plurality of actual rewards corresponding to the plurality of action sets respectively, a predicted value of the objective target volume, and an actual reward of the objective target volume; wherein each action set of the plurality of action sets comprises a target parameter combination composed of a plurality of different types of target parameters; and
generating the treatment plan for the to-be-treated target volume.

9. A non-transitory computer readable storage medium having stored a computer program thereon, wherein the computer program is used to implement a deep reinforcement learning model training method, wherein the deep reinforcement learning model is configured to include a plurality of actor network layers and a critic network layer, and different actor network layers of the plurality of actor network layers are configured to output different types of target parameters included in the treatment plan, wherein the method for training the deep reinforcement learning model for generating the treatment plan comprises:
performing a training process, the training process including the following operations:
acquiring initial dose distribution state data of an objective target volume;
determining, based on the initial dose distribution state data of the objective target volume, current policy data of the plurality of actor network layers, and current policy data of the critic network layer, target data; and
updating, based on the target data, the current policy data of the plurality of actor network layers and the current policy data of the critic network layer, so as to complete a current training for the deep reinforcement learning model; and
iterating the training process until a count of training the deep reinforcement learning model reaches a preset count, so as to obtain the deep reinforcement learning model that has been trained;
wherein the target data comprises: a final dose distribution of the objective target volume, multiple dose distribution state data of the objective target volume, a plurality of action sets output by the plurality of actor network layers corresponding to the multiple dose distribution state data respectively, a plurality of predicted values output by the critic network layer corresponding to the plurality of action sets respectively, a plurality of actual rewards corresponding to the plurality of action sets respectively, a predicted value of the objective target volume, and an actual reward of the objective target volume; wherein each action set of the plurality of action sets comprises a target parameter combination composed of a plurality of different types of target parameters.
